(19) United States
(12) Patent Application Publication
(10) Pub. No.: US 2020/0261058 A1
(43) Pub. Date: Aug. 20, 2020

(54) ULTRASOUND DIAGNOSTIC APPARATUS

(71) Applicant: KONICA MINOLTA, INC., Tokyo (JP)

(72) Inventor: Takahiko SHIRAISHI, Tokyo (JP)

(73) Assignee: KONICA MINOLTA, INC., Tokyo (JP)

(21) Appl. No.: 16/741,881

(22) Filed: Jan. 14, 2020

(30) Foreign Application Priority Data
Feb. 19, 2019 (JP) 2019-027255

(51) Int. Cl. A61B 8/00

(52) U.S. Cl. CPC A61B 8/4433; A61B 8/4444; A61B 8/4405

(57) ABSTRACT

A cart-type ultrasound diagnostic apparatus includes a recess portion disposed at an outer side surface of a casing that constitutes an apparatus body of the ultrasound diagnostic apparatus; a probe connector that is disposed in the recess portion and to which a connection terminal of an ultrasound probe is connectable; and a cover member disposed at the outer side surface of the casing so as to cover the recess portion. The cover member, in a state in which the connection terminal of the ultrasound probe is connected to the probe connector, covers the recess portion and allows a cable of the ultrasound probe to be led out from inside of the recess portion to outside of the recess portion.

### CROSS REFERENCE TO RELATED APPLICATIONS

The entire disclosure of Japanese Patent Application No. 2019-027255 filed on Feb. 19, 2019 is incorporated herein by reference in its entirety.

### BACKGROUND

### Technological Field

The present invention relates to an ultrasound diagnostic apparatus.

### Description of Related Art

There is known an ultrasound diagnostic apparatus that transmits ultrasound to a subject and that generates an ultrasound image based on a reception signal obtained from an ultrasound echo from the inside of the subject.

This type of ultrasound diagnostic apparatus is demanded to be movable and installed at a location so that the ultrasound diagnostic apparatus can be used at any one of various locations. In the light of situations, an ultrasound diagnostic apparatus is suggested, the ultrasound diagnostic apparatus which includes a plurality of casters attached to a bottom portion of an apparatus body of the ultrasound diagnostic apparatus and is freely movable on the floor by artificial force (hereinafter, the apparatus is referred to as “cart-type ultrasound diagnostic apparatus”, for example, see Japanese Patent Application Laid-Open No. 2017-6476).

This type of ultrasound diagnostic apparatus, however, has a relatively large weight. When a user moves the ultrasound diagnostic apparatus, the user may strike an ambient obstacle (for example, a wall of a building) with the ultrasound diagnostic apparatus. In such a case, if a probe connector (or a connection terminal of an ultrasound probe connected to the probe connector) provided at an outer side surface of the ultrasound diagnostic apparatus is exposed to the outside, the probe connector (or the connection terminal of the ultrasound probe connected to the probe connector) may be damaged.

In particular, this type of ultrasound diagnostic apparatus in many cases has a portion serving as a grip (typically, an operation panel) when the user moves the ultrasound diagnostic apparatus and the portion is pivotal. The position of the probe connector may be in a blind spot of the user. In such a case, if the probe connector protrudes from the outer side surface of the ultrasound diagnostic apparatus, the risk of the user striking an obstacle with the probe connector further increases.

### SUMMARY

The present disclosure is made in the light of the above-described disadvantages and provides a cart-type ultrasound diagnostic apparatus capable of avoiding a damage on a probe connector and/or a connection terminal of an ultrasound probe due to a strike against an obstacle.

To achieve at least one of the abovementioned objects, according to an aspect of the present invention, a cart-type ultrasound diagnostic apparatus reflecting one aspect of the present invention comprises a recess portion disposed at an outer side surface of a casing that constitutes an apparatus body of the ultrasound diagnostic apparatus; a probe connector that is disposed in the recess portion and to which a connection terminal of an ultrasound probe is connectable; and a cover member disposed at the outer side surface of the casing so as to cover the recess portion, wherein the cover member, in a state in which the connection terminal of the ultrasound probe is connected to the probe connector, covers the recess portion and allows a cable of the ultrasound probe to be led out from inside of the recess portion to outside of the recess portion.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 illustrates a schematic configuration of an ultrasound diagnostic apparatus according to an embodiment;

FIG. 2 is a block diagram illustrating a main part of a control system of the ultrasound diagnostic apparatus according to the embodiment;

FIG. 3 illustrates an appearance of an ultrasound probe according to the embodiment;

FIG. 4 is a perspective view illustrating an appearance of an apparatus body according to the embodiment;

FIG. 5 is a perspective view illustrating the appearance of the apparatus body according to the embodiment;

FIG. 6 is an enlarged view illustrating an appearance of an outer side surface of a casing according to the embodiment;

FIG. 7 is an enlarged view illustrating the appearance of the outer side surface of the casing according to the embodiment; and

FIG. 8 is an enlarged view illustrating an attached state of a cover member according to the embodiment.

### DETAILED DESCRIPTION OF EMBODIMENTS

Hereinafter, one or more embodiments of the present invention will be described with reference to the drawings. However, the scope of the invention is not limited to the disclosed embodiments.

### Configuration of Ultrasound Diagnostic Apparatus

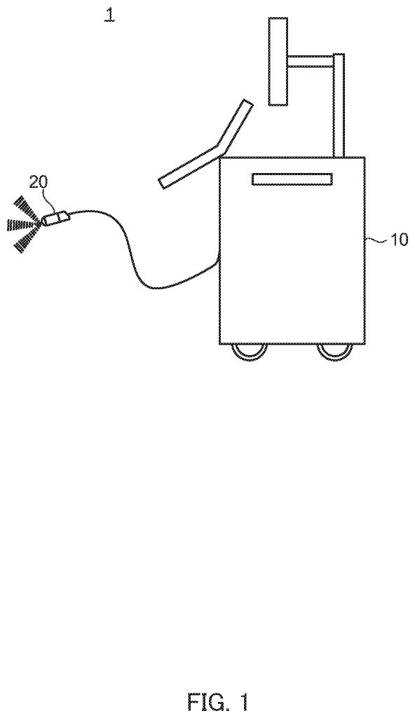

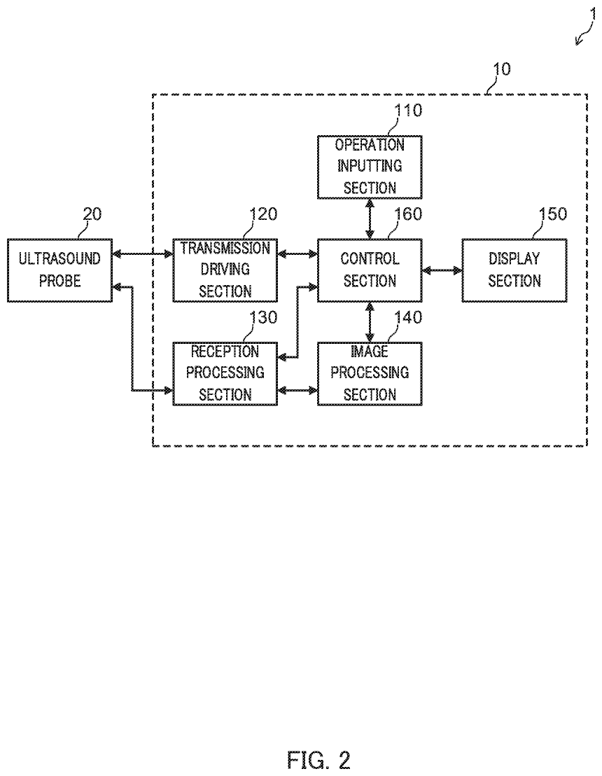

Hereinafter, an example of a configuration of ultrasound diagnostic apparatus 1 according to an embodiment is described with reference to FIGS. 1 and 2.

FIG. 1 illustrates a schematic configuration of ultrasound diagnostic apparatus 1 according to the embodiment. FIG. 2 is a block diagram illustrating a main part of a control system of ultrasound diagnostic apparatus 1 according to the embodiment.

Ultrasound diagnostic apparatus 1 includes apparatus body 10 and ultrasound probe 20, and is constituted by connecting ultrasound probe 20 to apparatus body 10.

Ultrasound diagnostic apparatus 1 is used to visualize as an ultrasound image the shapes, properties, and dynamic behaviors of the inside of a subject, and to use the ultrasound image for an image-based diagnosis. Ultrasound diagnostic apparatus 1 may generate a desirable ultrasound image, such as a B mode image, a color Doppler image, a three-dimensional ultrasound image, or an M mode image. Likewise, a desirable probe, such as a convex probe, a linear probe, a sector probe, or a three-dimensional probe, may be applied to the ultrasound probe 20.

Ultrasound probe 20 transmits ultrasound to a subject, receives an ultrasound echo reflected by the subject, converts the ultrasound echo into a reception signal, and transmits the reception signal to apparatus body 10.

Apparatus body 10 includes operation inputting section 110, transmission driving section 120, reception processing section 130, image processing section 140, display section 150, and control section 160.

Operation inputting section 110 receives, for example, an input of a command to instruct start of a diagnosis, or an input of information relating to the subject. Operation inputting section 110 receives inputs from, for example, an operation panel, a keyboard, and a mouse each including a plurality of input switches.

Transmission driving section 120, in response to an instruction of control section 160, generates a transmission signal and transmits the transmission signal to ultrasound probe 20.

Reception processing section 130 acquires a reception signal generated by ultrasound probe 20, performs reception processing (for example, phasing addition processing and filtering processing) on the reception signal, and then outputs the processed reception signal to image processing section 140.

Image processing section 140, in response to an instruction of control section 160, performs predetermined signal processing (for example, logarithmic compression processing, detection processing, and fast Fourier transform (FFT) analysis processing) on the reception signal acquired from reception processing section 130, and generates an ultrasound image (for example, a B mode image, a color Doppler image, or a three-dimensional ultrasound image) indicating the inside state of the subject. The contents of processing to generate an ultrasound image are known, and hence the description thereof is omitted.

Display section 150 displays the ultrasound image generated by image processing section 140. Display section 150 displays the ultrasound image on, for example, a liquid crystal display.

Control section 160 controls the entirety of ultrasound diagnostic apparatus 1 by controlling operation inputting section 110, transmission driving section 120, reception processing section 130, image processing section 140, and display section 150 in accordance with their respective functions.

### External Characteristics of Ultrasound Diagnostic Apparatus

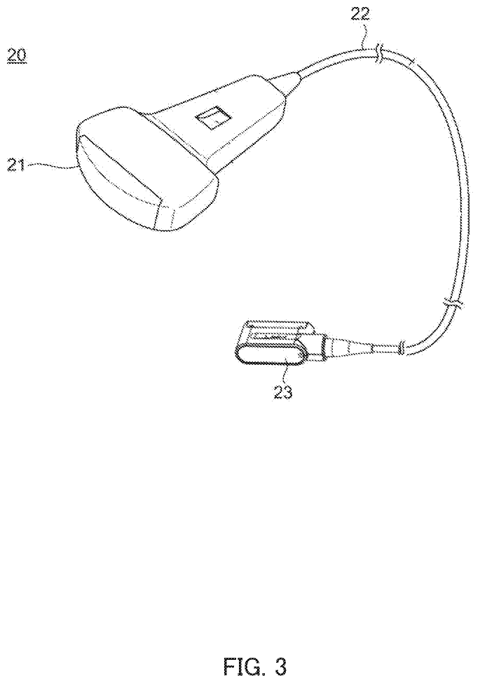

Next, external characteristics of ultrasound diagnostic apparatus 1 according to the embodiment are described with reference to FIGS. 3 to 8.

FIG. 3 illustrates an appearance of the ultrasound probe 20 according to the embodiment.

Ultrasound probe 20 includes an element array housed in a case. The element array includes piezoelectric elements arrayed in a one-dimensional direction or two-dimensional directions.

Ultrasound probe 20 includes probe body 21 including the element array and other components covered with a resin cover, connection terminal 23, and cable 22 that electrically connects probe body 21 to connection terminal 23.

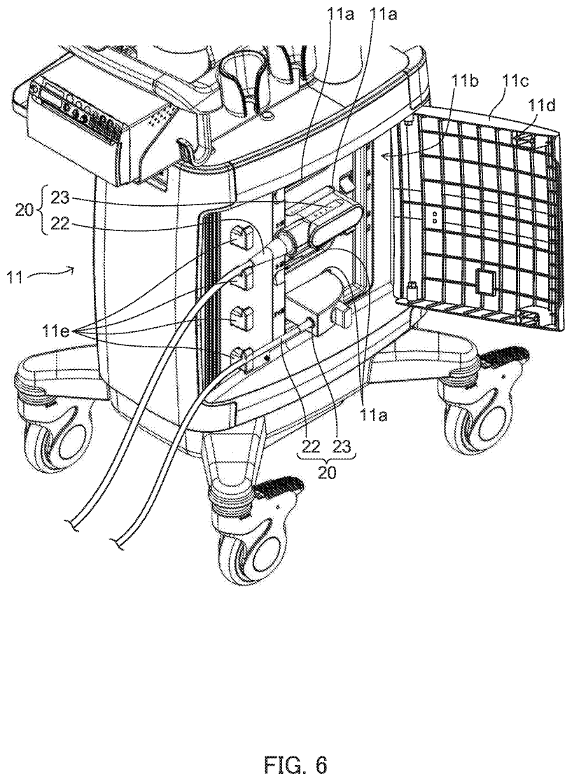

During an ultrasound inspection, ultrasound probe 20 is used in a state in which connection terminal 23 is connected to a probe connector (denoted by reference sign 11a in FIG. 6, described later) provided at apparatus body 10 and probe body 21 is held by the user.

Ultrasound probe 20 may be any one of various types of probes, such as a convex probe or a linear probe, which is prepared in accordance with the subject and purpose of an inspection.

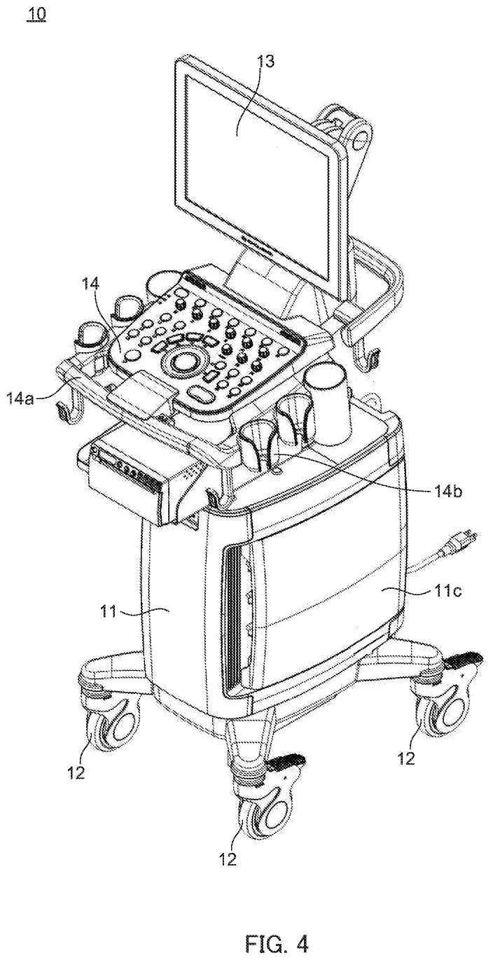

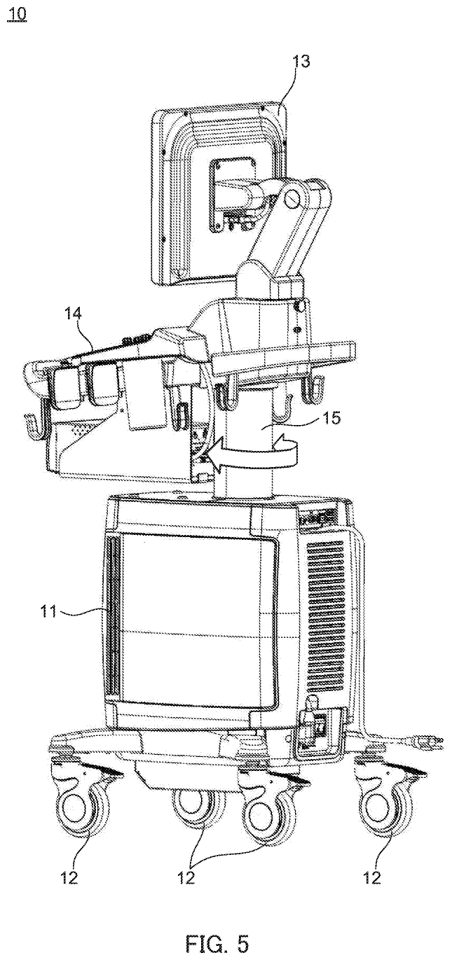

FIGS. 4 and 5 are perspective views each illustrating an appearance of apparatus body 10. FIG. 4 is a perspective view when apparatus body 10 is viewed from a front surface side. FIG. 5 is a perspective view when apparatus body 10 is viewed from a rear surface side.

Apparatus body 10 includes casing 11, caster 12 disposed at a bottom portion of casing 11, display 13 disposed above casing 11, and operation panel 14 disposed above casing 11.

Casing 11 is a housing box that houses a circuit board (not illustrated) that constitutes transmission driving section 120, reception processing section 130, image processing section 140, and control section 160. Casing 11 has, for example, a substantially rectangular-parallelepiped shape.

Caster 12 is provided at the bottom portion of casing 11, and causes apparatus body 10 to be movable relative to a floor surface. For example, caster 12 includes four casters 12 disposed at the four respective corners of the bottom portion of casing 11. Four casters 12 cause apparatus body 10 to be movable relative to the floor surface. Casters 12 each include a caster lock that constitutes a drum brake, and can be changed between a lock state and an unlock state through an operation by the user.

Display 13 displays an ultrasound image. Operation panel 14 receives an input operation from the user.

Display 13 and operation panel 14 are supported by support member 15 protruding vertically upward from the casing 11 (see FIG. 5). Display 13 and operation panel 14 are supported pivotally around a vertical axis relative to casing 11 via support member 15. Casing 11 includes a pivot mechanism (for example, a bearing mechanism) at a connection position to support member 15. The pivot mechanism supports support member 15 rotatably around the vertical axis relative to casing 11.

With the above-described configuration, display 13 and operation panel 14 are pivotal around the vertical axis relative to the casing 11 through a manual operation by the user (see an arrow in FIG. 5). That is, the user thus can pivot display 13 and operation panel 14 in accordance with the use environment. The pivot mechanism includes a lock member (not illustrated) that locks the rotation of support member 15. In an ordinary situation, the pivot mechanism is kept in a state of locking the rotation of support member 15. When the lock member unlocks the rotation of support member 15, the pivot mechanism allows display 13 and operation panel 14 to be pivotal.

Operation panel 14 includes handle 14a serving as a grip when operation panel 14 is rotated, and probe holder 14b to place ultrasound probe 20.

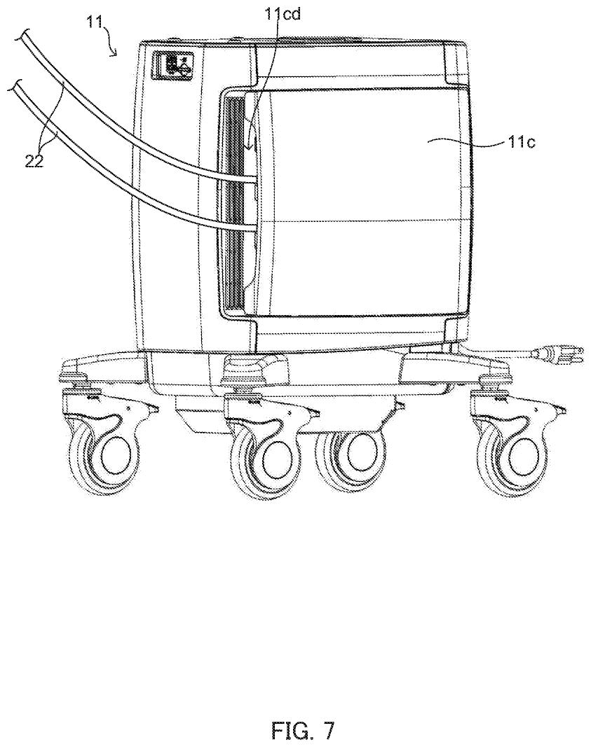

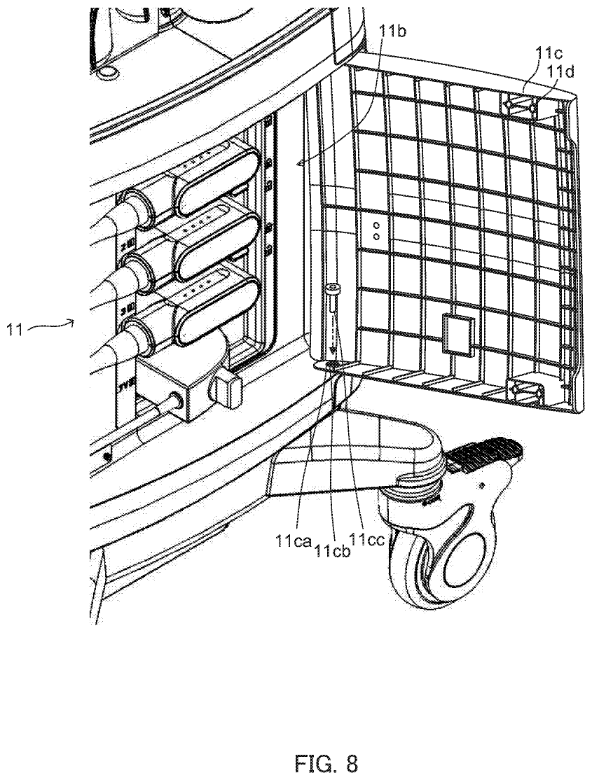

FIGS. 6 and 7 are enlarged views each illustrating an appearance of an outer side surface of casing 11. FIG. 6 illustrates a state in which a cover member 11c is open. FIG. 7 illustrates a state in which the cover member 11c is closed. FIG. 8 is an enlarged view illustrating an attached state of cover member 11c.

Casing 11 has probe connector 11a, recess portion 11b that constitutes a housing portion of probe connector 11a, cover member 11c, magnet 11d, and cable hook 11e.

Probe connector 11a is a connector to which connection terminal 23 of ultrasound probe 20 is electrically connected. That is, probe connector 11a relays transmission and reception of electric signals between ultrasound probe 20 and transmission driving section 120 (and reception processing section 130) of apparatus body 10.

Probe connector 11a is provided in recess portion 11b provided at the outer side surface of casing 11. In the embodiment, probe connector 11a includes four probe connectors 11a disposed in recess portion 11b. FIG. 6 illustrates a state in which connection terminals 23 of different ultrasound probes 20 are connected to second probe connector 11a from the top and fourth probe connector 11a from the top among four probe connectors 11a.

Recess portion 11b is provided at the outer side surface of casing 11, and defines a housing space that houses probe connectors 11a. Recess portion 11b has a shape and a size that completely house probe connectors 11a therein. Recess portion 11b typically has a depth so that upper end portions of probe connectors 11a do not protrude from the outer side surface of casing 11. More preferably, recess portion 11b has a depth so that, when connection terminal 23 of ultrasound probe 20 is connected to probe connector 11a, an upper end portion of connection terminal 23 is located at a position retracted from the outer side surface of casing 11.

Cover member 11c is attached to the outer side surface of casing 11 so as to cover recess portion 11b. Cover member 11c covers the entirety of recess portion 11b so that probe connector 11a and connection terminal 23 of ultrasound probe 20 connected to probe connector 11a are not exposed to the outside of recess portion 11b.

Cover member 11c is typically an openable and closable panel of recess portion 11b and is attached to the outer side surface of casing 11. That is, cover member 11c changes the state of recess portion 11b from a state covered with the cover member 11c to a state not covered with the cover member 11c and exposed to the outside of casing 11 through a manual operation by the user. Cover member 11c is typically in the open state when ultrasound probe 20 is connected to probe connector 11a to allow the user to easily perform a connecting work. Cover member 11c is in the closed state after ultrasound probe 20 is connected to probe connector 11a to protect probe connector 11a and/or connection terminal 23 of ultrasound probe 20.

Cover member 11c is openable and closable, for example, by a hinge structure including through hole 11ca provided in the cover member 11c, a through hole 11cb provided in the outer side surface of the casing 11, and a pin 11cc inserted through through holes 11ca and 11cb (see FIG. 8). Pin 11cc is removable. The cover member 11c is removably attached to casing 11 by pin 11cc.

Cover member 11c has cable lead-out port 11cd to lead out cable 22 of ultrasound probe 20 from the inside of recess portion 11b to the outside of recess portion 11b (see FIG. 7). Cable lead-out port 11cd is, for example, a depression portion provided at an end portion at a lateral side of cover member 11c, and allows cable 22 of ultrasound probe 20 to be led out through a gap formed between cover member 11c and the outer side surface of casing 11. Thus, recess portion 11b can be covered with cover member 11c in a state in which connection terminal 23 of ultrasound probe 20 is connected to probe connector 11a.

Cover member 11c has an outer exposed surface having a shape, in a state in which cover member 11c covers recess portion 11b, so that the outer exposed surface is flush with the outer side surface of casing 11 at the periphery of recess portion 11b, and the outer exposed surface has the same flat surface or the same curved surface as the outer side surface of casing 11. Thus, cover member 11c can be visually recognized as part of the outer side surface of casing 11. The design of ultrasound diagnostic apparatus 1 can be improved.

Magnet 11d attracts cover member 11c toward a side surface of casing 11 by using magnetic force to stably keep the state in which cover member 11c covers recess portion 11b (see FIG. 6). Magnet 11d is disposed at, for example, an end portion on the side opposite to the position of the hinge structure of cover member 11c. Magnet 11d exhibits an attraction effect with respect to a stainless steel member (for example, a screw member) provided at the outer side surface of casing 11. Magnet 11d may be disposed at cover member 11c, may be disposed at the outer side surface of casing 11, or may be disposed at both.

Cable hook 11e is disposed in recess portion 11b, and hooks cable 22 of ultrasound probe 20 extending from probe connector 11a. Cable hook 11e is disposed, for example, at a position at which cable 22 of ultrasound probe 20 extends from the side of probe connector 11a when ultrasound probe 20 is connected to probe connector 11a.

Since cable 22 of ultrasound probe 20 is hooked to cable hook 11e, sagging of cable 22 is reduced. This can prevent occurrence of a phenomenon in which cable 22 of ultrasound probe 20 sags, and hence cable 22 is stepped on by caster 12 or cable 22 is caught in cover member 11c when cover member 11c is closed.

When ultrasound diagnostic apparatus 1 according to the embodiment is used, the user first opens cover member 11c and connects connection terminal 23 of ultrasound probe 20 to probe connector 11a disposed in recess portion 11b. Then, the user leads out the cable 22 and the probe body 21 of the ultrasound probe 20 to the outside of recess portion 11b in the state in which cable 22 of ultrasound probe 20 is hooked to cable hook 11e. The user brings cover member 11c into the closed state. In such a state, the user executes an ultrasound inspection by using ultrasound probe 20.

### Advantageous Effects

As described above, with ultrasound diagnostic apparatus 1 according to the embodiment, even when the user strikes an obstacle with ultrasound diagnostic apparatus 1 when the user moves ultrasound diagnostic apparatus 1, cover member 11c can reduce a shock against probe connector 11a and/or connection terminal 23 of ultrasound probe 20. Accordingly, a damage on probe connector 11a and/or connection terminal 23 of ultrasound probe 20 can be avoided.

In addition, accordingly, the region where probe connector 11a and/or connection terminal 23 of ultrasound probe 20 is disposed can be covered. The design of the appearance of ultrasound diagnostic apparatus 1 can be improved.

Although embodiments of the present invention have been described and illustrated in detail, the disclosed embodiments are made for purpose of illustration and example only and not limitation. The scope of the present invention should be interpreted by terms of the appended claims.

With the cart-type ultrasound diagnostic apparatus according to the present disclosure, a damage on the probe connector and/or the connection terminal of the ultrasound probe can be avoided.

What is claimed is:

1. A cart-type ultrasound diagnostic apparatus, comprising:
a recess portion disposed at an outer side surface of a casing that constitutes an apparatus body of the ultrasound diagnostic apparatus;
a probe connector that is disposed in the recess portion and to which a connection terminal of an ultrasound probe is connectable; and
a cover member disposed at the outer side surface of the casing so as to cover the recess portion, wherein
the cover member, in a state in which the connection terminal of the ultrasound probe is connected to the probe connector, covers the recess portion and allows a cable of the ultrasound probe to be led out from inside of the recess portion to outside of the recess portion.

2. The ultrasound diagnostic apparatus according to claim 1, wherein the cover member is attached to the casing in an openable and closable manner.

3. The ultrasound diagnostic apparatus according to claim 2, further comprising:
a magnet that is attached to the cover member or the casing and that keeps a state in which the cover member covers the recess portion by using magnetic force.

4. The ultrasound diagnostic apparatus according to claim 1, wherein the cover member is removably attached to the casing.

5. The ultrasound diagnostic apparatus according to claim 1, wherein the cover member has an outer exposed surface having a shape, in a state in which the cover member covers the recess portion, so that the outer exposed surface is flush with the outer side surface of the casing at a periphery of the recess portion.

6. The ultrasound diagnostic apparatus according to claim 1, wherein the connection terminal of the ultrasound probe is located at a position retracted from the outer side surface of the casing at a periphery of the recess portion in the state in which the connection terminal is connected to the probe connector.

7. The ultrasound diagnostic apparatus according to claim 1, further comprising:
an operation panel provided above the casing and attached pivotally around a vertical axis relative to the casing.

8. The ultrasound diagnostic apparatus according to claim 1, further comprising:
a cable hook that is provided in the recess portion and that hooks the cable of the ultrasound probe.
